(19) United States
(12) Patent Application Publication
(10) Pub. No.: US 2012/0237555 A1
(43) Pub. Date: Sep. 20, 2012

(54) SURFACE COATING FOR BIOMOLECULE IMMOBILISATION AND MINIMISATION OF NON-SPECIFIC BINDING ON SURFACES FOR BIOMEDICAL DIAGNOSTICS

(76) Inventors: David Williams, St. Heliers (NZ); Vladimir Gubala, Dvomiky (SK); Ram Prasad Gandhiraman, Tamilnadu (IN); Christy Charlton, White Lake, MI (US); Stephen Daniels, Dublin (IE); Nam Cao Hoai Le, TP Bao Loc (VN)

(21) Appl. No.: 13/049,086

(22) Filed: Mar. 16, 2011

(51) Int. Cl. G01N 33/53; A61K 9/00; H05H 1/24; C12Q 1/02; C12M 1/00

(52) U.S. Cl. 424/400; 436/501; 435/29; 435/283.1; 427/578; 427/2.11; 427/2.14

(57) ABSTRACT

A solid substrate coating capable of swelling by a factor of at least 2 on contact with an aqueous solution. The solid substrate coating may be prepared by activating the surface of a solid substrate by treatment with a plasma, depositing a first layer of siloxane onto the surface of the solid substrate using plasma, and depositing a second layer of at least on chemical functionality on top of the first layer using plasma.

### FIELD OF THE INVENTION

The invention relates to biocompatible siloxane based films or surface coatings produced on a polymer or polymer-like substrate by plasma-enhanced chemical vapour deposition (PECVD). The deposited films or coats display high non-specific binding in biomolecule immobilisation and biomedical diagnostic applications, where minimisation of non-specific binding on surfaces is desirable.

### DESCRIPTION OF RELATED ART

Recently much interest has been shown in a new class of thermoplastic polymer, cyclic olefin copolymer (COC). Such polymers are of interest because of their favourable properties, such as high glass transition temperature, low autofluorescence, optical clarity, resistance to organic solvents, low water uptake and mouldability^{1,2} Zeonor® is one example of an amorphous polymeric material or COC having these desirable properties, whilst providing a cost effective and disposable platform for biodiagnostic devices.

COCs are pure hydrocarbons and thus possess no native groups amendable to covalent functionalization. For covalent attachment or immobilization of biomolecules, for biosensor applications or for use in biomedical diagnostic devices, for example, the plastic surface needs to be functionalised to facilitate bioanalyte attachment. For such devices to work efficiently, the biomolecules need to be immobilised on surfaces in their biologically active state with low non-specific binding.

Functionalization of surfaces through liquid phase deposition of reactive groups (carboxy, amine or aldehyde functional groups, for example) is a routinely used procedure for immobilizing the biomolecules for biosensors applications. However, this process has several drawbacks, for example, a water free environment is needed, unwanted polymerisation can occur, the process is time consuming and requires hazardous materials³. Further more, the liquid phase deposition process is not ideal for large-scale industrial production. Furthermore, known films, such as the aminosilane film of International Publication WO2006/085898 show high non specific binding, especially for negatively charged molecules (see FIG. 4 below).

Therefore, it would be desirable to provide substrates or coated substrates which are suitable for industrial scale production, that may be used in biodiagnostic devices which require a high analyte binding capacity, with low non-specific binding, but also show good analyte adhesion, surface stability and resistance against the washing and regeneration conditions as well a high signal to noise ratio.

### SUMMARY OF THE INVENTION

According to the present invention, as set out in the appended claims, there is provided a method of preparing a solid substrate coating that swells on contact with an aqueous solution by factor of at least 2, based on a RMS (root mean square) roughness swelling measurement, the method comprising the steps of:
(i) activating a surface of the solid substrate by treatment with a plasma;
(ii) depositing a first layer of siloxane onto the surface of the solid substrate using plasma; and
(iii) depositing a second layer of at least one chemical functionality on top of the first layer using plasma.

The level of the plasma power is selected so as to activate the surface of the solid substrate by providing reactive oxygen and/or hydroxyl containing species on the surface while supplying sufficient energy to promote the chemical reactions that form an adherent polymerised surface layer but without excessive fragmentation of the chemical functionality deposited in step (iii). The required plasma power can conveniently be monitored through mass spectrometric observation of the chemical species present in the plasma^{4,5}.

The coated substrates of the invention have been found to possess several properties that make them particularly suitable for use in bioanalytical techniques. The specific characteristics of the coatings of the invention have been found to be highly dependent on the nature of the solid substrate, the chemical precursors and plasma process parameters, particularly the applied input power and deposition time. Thus the method of invention provides a technique where the properties of the substrate coating are tailorable. It has been found that the use of specific optimised plasma deposition and treatment parameters ensure that the substrate surface displays the most desirable qualities for use in biological assay applications. Important qualities such as improved adhesion, spreading and proliferation of cells at the surface, as well as to improved membrane properties such as hydrophobic/philic character, non fouling properties, transport and capability of immobilising molecules⁶ are all controllable. Such alterations are controlled by PECVD process parameters, and include as gas flow rate, chamber pressure, deposition time and input power to gas mixture. It has been found to that solid substrate coating formed by the method of the present invention under the influence of a plasma polymerisation conditions, (i) possess a high degree of reactive functionality for binding of biorecognition elements and bioanalyte targets; (ii) facilitate retention of the specifically bound targets (such Co antibodies/nucleic acids) to a high degree; and (iii) minimize the general non-specific binding (NSB) of non-analyte constituents of the sample. Non-specific binding varies depending on (i) the way the chemical functionalities are plasma polymerised and (ii) on the nature of underlying layer on which the chemical functional groups are functionalised and (iii) on the water absorption characteristics of the coating. The swelling of the plasma polymerized layer upon exposure to aqueous solution affects the way the biomolecules interact with the surface and significantly alter the non-specific binding in favour of non specific binding.

Furthermore and advantageously, it has been found that the substrate coating of the invention retains adhesion with the surface of the substrate after exposure to biological solutions for at least 4 hours. Thus, the substrate film or coating of the invention is suitable for immobilising analytes, such as capture antibodies while minimising non-specific adsorption of labelled antibodies to the capture antibodies. Accordingly, the plasma prepared surfaces may be used in biodiagnostics applications, where immobilization of biomolecules plays a key role in the performance of the bioassay devices.

In this technique, the method is an in-situ process, a plasma discharge is created in presence of chemical precursor containing the required chemical group for deposition or deposition and functionalization. Suitably, the plasma arises from plasma enhanced chemical vapour deposition technique (PECVD). Plasma enhanced chemical vapour deposition is a completely dry process and is more appropriate for bulk industrial manufacturing compared to that of normally used wet chemical process. Thus the method is typically carried out under solvent free conditions. The plasma layer deposition technique has a number of advantages over multistep, wet chemical methods or CVD used for layer deposition. PECVD can be used to coat a large number of substrates. PECVD avoids direct contact with solvent, thus reducing chemical waste and importantly, it operates at room temperature maintaining required functionality.

In this method, the siloxane is deposited by plasma polymerisation of a siloxane precursor chemical compound and the at least one chemical functionality is deposited by plasma polymerisation of at least one chemical functionality precursor chemical compound. Thus, the siloxane layer of step (i) and the chemical functionality layer of step (ii) may be deposited by action of the plasma on suitable precursor chemical compounds. As stated above, the optimum plasma power conditions required for the method of the invention may vary from instrument to instrument depending on factor known to the person skilled in the art, such as operating pressure, plasma gas used, operating temperature, degree of ionization within the plasma, electrode configuration, magnetic field etc. The optimum plasma power for any given instrument may be determined by measuring characteristics of the plasma produced by the instrument. For example, a mass-spectrometric method may be used for mass-spectrometric analysis of at least one precursor in the plasma in order to identify the conditions needed for any particular apparatus in order to identify the required minimum power to achieve sufficient fragmentation of the precursors. In the present case, the optimum plasma power for any given instrument may be determined by, for example, collecting a mass spectroscopic fragmentation profile of the desired chemical precursor to the siloxane and/or to the chemical functionality in the plasma. The plasma power may then be adjusted such that the fragmentation of chemical functional groups of the chemical functionality precursor is avoided. However, it is important to have a minimum power that can activate the substrate surface and to supply sufficient energy to promote the chemical reactions that form an adherent polymerised surface layer. The required power is that which is just sufficient to cause a small degree of fragmentation of the precursors (typically a 20% reduction in the amount of un-fragmented precursor). For example, FIG. 3 indicates the optimum plasma power for a siloxane precursor (TEOS) and a chemical functionality precursor (acrylic acid) in an argon plasma (see FIG. 3, where carbonyl has atomic mass unit 28 and carboxyl group has atomic mass unit 44). Thus the plasma power can be determined by adjusting the plasma parameters to produce a mass spectroscopic fragmentation pattern corresponding to one of patterns of siloxane precursor or a chemical functionality precursor as shown in FIG. 3.

The precise power and deposition time allow the formation of coatings in which the physical coating properties are highly tailorable to a particular application. Preferably, the method step (i) involves activating the surface of the solid substrate by treatment with a plasma activation step at a plasma Radio Frequency (RF) power from about 20 Watts to about 300 Watts.

Surface activating step by plasma (i) prepares the solid substrate surface by, for example, removing any contaminants, and providing reactive groups, such as hydroxyl groups, on or near the solid substrate surface. The plasma pre-treatment or plasma activation process step is carried out this relatively high plasma power as stated above relative to the lower power of step (iii), so as to introduce highly reactive oxygen and or hydroxyl containing species onto the surface of the polymer or polymer like substrate. These species are required to facilitate adherence of the siloxane layer of step (ii).

Step (ii), involving the subsequent introduction of a siloxane precursor or siloxane-containing derivative under action of the plasma, results in a strongly adherent layer formed on the solid substrate surface resulting from covalent siloxane bonds to highly reactive oxygen and or hydroxyl containing species formed on the polymer or polymer like substrate surface material in step (i). The siloxane film forms under the action on the plasma on a siloxane precursor which may be a siloxane or an oxygen containing silane.

Step (iii) allows the chemical functionality precursor to cross-link and polymerize on the newly formed siloxane layer surface where the siloxane and the chemical functionality precursor crosslink and reacts to form functionalised reactive surface built on top of the siloxane first layer. The network of at least one chemical functionality thus formed results from the action of the plasma on the chemical functionality precursor^{7,8}.

Preferably, the method step (ii) involves depositing the first film of siloxane onto the solid substrate surface by a deposition method at a plasma Radio Frequency (RF) power of from about 5 Watts to about 300 Watts.

Preferably, method step (iii) involves depositing the network of at least one chemical functionality on top of the siloxane film of the solid substrate surface by a deposition method at plasma Radio Frequency (RE) power from about 5 Watts to about 100 Watts.

In a preferred embodiment, step (i) is carried out for a period of from about 30 seconds to about 5 minutes. Suitably, the deposition time of step (ii) is preferably from about 5 seconds to about 30 minutes. Suitably, the deposition time for step (iii) is from about 5 seconds to about 30 minutes.

However, in some embodiments, shorter deposition times of from about 5 seconds to about 5 minutes for step (ii) and from about 5 seconds to 5 minutes for step (iii) are particularly desired, since this leads to the formation of coatings with higher binding capacity and lower non-specific binding properties.

In the case, where siloxane is used as adhesion and network building layer in step (ii) and acrylic acid as the chemical functionality precursor in step (iii), a deposition of step (ii) is preferably about 15 seconds, and the deposition step of (iii) is about 30 seconds is particularly preferred as a coating with desirable properties arises. In a particularly preferred embodiment involving siloxane and AA, step (i) may be carried out at a Radio Frequency (RF) power from about 20 Watts to about 300 Watts, step (ii) may be carried at a Radio Frequency (RF) power plasma energy from about 5 Watts to about 300 Watts in step (ii), and step (iii) may be carried out at a RF power of from about 5 Watts to about 100 Watts. These conditions are particularly suited to use of siloxane and adhesion and network building layer in step (ii) and acrylic acid as the chemical functionality precursor in step⁹ (iii) as a coating showing particularly favorable properties is formed.

In which the surface of the solid substrate is (i) pre-treated or plasma activated by treatment with a mixed plasma mixture of a noble gas such as argon or helium, with oxygen or water vapour at a (high) Radio Frequency (RF) power of from about 20 Watts to about 300 Watts, followed by subsequent exposure to vapours of a siloxane precursor such as siloxane or an oxygen rich silane, at a plasma power of from about 5 Watts to about 300 Watts in step (ii) and further exposure to vapour of a functional group precursor with the desired functional groups at a (lower) RF power of from about 5 Watts to 100 Watts during step (iii). The relatively high input RF power applied during the plasma activation step (i) creates highly reactive oxygen and or hydroxyl containing species such as radicals and ions at the substrate surface, while the application of very low RF power of step (iii) during the plasma polymerization process retains the chemical functionality while stopping the layer from fragmenting at high applied input power.

Examples of suitable functional group precursor compounds include amines, ethers, thiols and per-fluoro compounds. In particular, amine surface functionalization may result from using aminopropyltriethoxysilane (APTES) or alternatively mixture of APTES and ethylenediamine (EDA)¹⁰. Thiol surface functionalization may result using from mercaptopropyltriethoxysilane (MPTES), epoxy surface functionalization arises from use of 3-glycidoxypropyltrimethoxysilane (GOPTMS), perfluorine surface functionalization arises from perfluoroctyltriethoxysilane (FOTES)¹¹, carboxylic acid functionality may arises from co-deposition of tetraorthosilicate (TEOS), hexamethyldisiloxane (HMDSO)¹², acrylic acid (AA), diethyleneglycol dimethylether (DEGDME i.e. PEG) and their combinations, aldehyde surface functionality from co-deposition of TEOS, HMDSO, acetaldehyde and formaldehyde, and alcohol functionality may arise from co-deposition of TEOS, HMDSO, acetaldehyde, PEG. In a preferred embodiment, the coating may be prepared by using one single chemical functionality precursor compound that containing both the siloxane group and the functional group (FIG. 1, left). Suitable precursors include aminopropyltriethoxysilane (APTES) mercaptopropyltriethoxysilane (MPTES), 3-glycidoxypropyltrimethoxysilane (GOPTMS), perfluoroctyltriethoxysilane (FOTES) etc.

In a preferred embodiment, method steps (ii) and (iii) above may be carried out simultaneously in a co-deposition step. That is, the siloxane precursor and the chemical functionality precursor are codeposited through plasma polymerisation onto the solid substrate surface to form the desired coating of the invention in simultaneous co-deposition step. Thus, co-deposition of the siloxane network building layer can be achieved by using a siloxane selected from the group comprising: tetraorthosilicate (TEOS), trimethoxysilane (TMS) and hexamethyldisiloxane (HMDSO), followed by deposition of volatile precursors such as acrylic acid (AA), acetic acid (AcA), acetaldehyde (AH), formaldehyde (FH) or diethyleneglycol dimethyl ether (DEGDME, further referred as PEG) can be used. An advantage of the co-deposition process is the simplicity and low cost of a large range of readily available precursors. Co-deposition of volatile glycol-ether compounds can advantageously be used to add poly(ethylene oxide)-like functionality to the layer.

Control of the plasma process parameters is necessary to ensure that the surface is rich in the desired functionalities, and to ensure that these functionalities are not completely broken down in the highly energetic plasma. Such plasma parameter optimizations are known in the art, for example, a mass spectroscopic analysis or screening step may be used to determine the optimum plasma conditions. Thus in a preferred embodiment, the method described above is preceded by an optional plasma parameter screening step. Suitable the screening step may be a mass spectroscopic step in which the mass to charge profile of the plasma is analysed to determine the optimum conditions needed.

In general the operating pressure of the plasma instrument preferably ranges from about 80 mTorr to about 600 mTorr. More preferably, the desired operating pressures may be selected from about 80 mTorr to about 250 mTorr.

Optionally, the addition of an oxygen or carbon source to the plasma, for example, for an oxygen source, water, ethanol, methanol, and for a carbon source are toluene, hexane) which can serve to enhance the functionality of the layer, build the network or to induce mixing or polymerization of the layer. However, since the method is preferably carried out under solvent free conditions, then the vapors of these additives are added to the plasma process during the above described method.

Another advantage stems from the fact that the method of the invention allows independent control the chemical functionality and hydrophilicity of the surface, which can be achieved by either sequential or co-depositions combined with adjustment of the plasma in order to achieve a graded ion induced mixing of the surface by bombardment of the plasma.

Preferably, the solid substrate or the solid substrate surface is a polymer, silicon, gold or glass. A solid substrate is selected from the group consisting of a plastic, a gold or silver metal is preferred. Plastic is the most preferred.

More preferably still, the solid substrate or the solid substrate surface is a hydrophobic polymer. Suitably, the polymer may be a plastics polymer, such cyclo olefin copolymer, polycarbonates, polydimethylsiloxane (PDMS) and poly(methyl methacrylate) (PMMA). Suitably, a cyclic olefin polymer is preferred, and for example may be Zeonor™®, Zeonex™®, Topas™. Suitable metal surfaces include gold, silver, and semiconducting surfaces like silicon, siliconoxynitride, gadolinium oxide. Erbium oxide, Hafnium oxide, titanium oxide glass and ceramic surfaces. Preferably, the solid substrate is a material selected from the group consisting of: a plastic, gold or silver metal. Suitable solid substrates also include plastic substrates coated with gold or silver and glass substrates coated with gold or silver.

Suitably, the at least one chemical functionality precursor is a compound having a chemical functionality selected from the group consisting of: carboxyl, ether, acrylic acid, acetic acid, acetaldehyde, formaldehyde, amines, ethers, thiols and per-fluoro functionality. Most preferably, the precursor to the at least one chemical functionality comprises carboxylate or ether or amine functionalities alone or in combination. If ether functionality is desired, it may be incorporated by co-deposition using volatile glycol-ether precursor, such diethylene glycol dimethylether (DEGDME). The preferred reactive functionality may be selected from the group consisting of: carboxyl, ether, acrylic acid, acetic acid, acetaldehyde and formaldehyde. Carboxyl functionality is most preferred, as the surface carboxyl groups may activated by known techniques, for example, EDC/NHS chemistry. The surface carboxyl functional groups server as a reactive handle to immobilize biomolecules of interest, if desired, onto the substrate. Thus, suitably, the chemical functionality arises from carboxylic, acrylic acid (AA), acetic acid, acetaldehyde, formaldehyde or other oxygen containing precursor compounds. Acrylic acid functionality is also preferred as it has been found that plasma polymerization of acrylic acid onto siloxane network leads to formation of a highly hydrophilic film containing a high number of carboxy groups, which are advantageously predominately negatively charged under conditions of physiological pH. This effectively creates a large hydration sphere and acts as a non-fouling layer to repel biomolecules that are also negatively charged, for example, DNA or proteins with a low isoelectric point.

In particular, a preferred combination arises from the use of siloxane as adhesion and network building layer and use of carboxy functionalization in the second film or layer, as the Inventors have found that the non-specific binding is surprisingly significantly reduced using this combination. Thus, carboxy functional groups can be deposited on substrates, such as cyclic olefin polymer (COP) and used in biomolecule immobilization applications. Advantageously, the activated carboxy functional groups can be linked to the biomolecules such as DNA, antibodies, proteins etc., directly, without the use of any cross-linker.

Suitably, the plasma polymerised substrate coatings prepared by this method are graded in composition, being silica-rich near the substrate surface and first layer interface and carbon-rich and highly functionalised near the outer surface of the second layer. The skilled person will appreciate that at the interface of the substrate surface and the first layer, and the interface between the first and second plasma polymerised layers, there may be a degree on intermingling of the layer arising from chemical binding between materials at the interface.

The method of the invention provides reactive surface coatings which have a water contact angle in the range of from about 5 degree to about 60 degrees, depending on the plasma and method parameters chosen. This is desirable, the surface coatings are hydrophilic and thus wettability is enhanced and flow of liquid into swellable surface is rapid. Both properties are advantages for bioassay devices.

Preferably, the method of the invention results in a substrate coating having a silicon content in the range of about 0.2 to about 15.00 atomic % as determined by XPS technique. Preferably having a silicon content in the range of about 0.25 to about 14.50 atomic % as determined by XPS. More preferably still, the silicon content in the coating is in the range of about 0.31 to 14.01 atomic % as determined by XPS. Suitably, the silicon content may be in the range of about 1 to about 13.00 atomic %, from about 5 to about 10.00 atomic %, from about 7 to about 9.00 atomic %.

The most preferred precursors are those that provide carboxyl functionality onto the deposited siloxane. Preferably the content of the carbon in the coating is in the range of from about 1 to about 86% atomic percent, more preferably the content of the carbon in the coating is in the range of from about 10 to about 76% atomic percent as determined by XPS, even more preferably the content of the carbon in the coating is in the range of from about 21.2 to about 59.3% atomic percent. Suitably, content of the to carbon in the coating may be in the range of about 15 to about 65 atomic %, from about 25 to about 45 atomic %, from about 30 to about 40 atomic %, as determined by XPS.

Preferably, surface carbonyl groups in the range of from about 0.5 to 50% as determined by XPS, more preferably having a surface carbonyl groups in the range of from about 5 to 30% as determined by XPS, more preferably having a surface carbonyl groups in the range of from about 5.5 to 28% as determined by XPS, more preferably still, from about 5% to 26% surface carbonyl groups as determined by XPS. Suitably, content of the surface carbonyl groups in the coating may be in the range of about 2.5 to about 45 atomic %, from about 10 to about 35 atomic %, from about 20 to about 30 atomic %, as determined by XPS.

The most preferred precursor to the at least one chemical functionality are acrylic acids. Acrylic acid is the preferred chemical functionality, since where the above method utilises acrylic acid, and siloxane, a plasma deposition period of about 15 s for step (ii) and about 30 s for step (iii) provide a substrate surface having particularly high binding capacity, the lowest non-specific binding and desirable water solubility properties.

The siloxane film functions as an adhesion layer and network building layer for further substrate functionalization with the desired volatile functional group precursor compounds which add functionality into the layer. It is preferred that the siloxane film is formed from tetraorthosilicate (TEOS) or hexamethyldisiloxane (HMDSO) or a mixture thereof. Particularly preferred is tetraorthosilicate TEOS. Silanes not comprising oxygen are not suitable as they do not provide surface with high non specific binding, nor do they swell in water.

The coating produced by the method of the invention results in a layer thickness is in the range of about 10 angstroms to about 1000 angstroms, preferably from about 20 angstroms to about 800 angstroms, more preferably from about 25 angstroms to about 600 angstroms, even more preferably from about 30 angstroms to about 400 angstroms.

The method of the invention allows substrate surface patterning can be achieved through use of a mask. A masking step involves a portion of the substrate surface being masked to facilitate substrate surface patterning.

The present method has provided surfaces which display surprisingly high non-specific binding. While plasma deposition of acrylic acid is known, the skilled person will appreciate that non-specific binding on plasma polymerised acrylic acid is very high. The water swellable solid substrate coating obtained directly from the method of the invention are also contemplated.

Thus, in a related aspect there is provided a composite substrate comprising a polymer or polymer-like surface having a first surface film comprising a siloxane network and a second surface film comprising a reactive functionality provided on top of the first surface film. Thus the method of the invention results in a solid substrate surface comprising a first film comprising a siloxane network and a second film or network comprising a reactive or activatable chemical functionality which is provided on top of the first film of siloxane.

In one aspect, there is provided a coating for a solid substrate, the coating comprising:

a first siloxane layer; and

a second layer comprising a chemical functionality

wherein the first and second layers are plasma polymerised onto the substrate, the coating swellable on contact with an aqueous solution by factor of at least 2, based on RMS (root mean square) roughness swelling measurement. RMS roughness may be measured by atomic force microscopy (AFM).

Suitably, the coating provides a ratio of specific to non specific analyte binding in bioassays of greater that 2 as detected using fluorescent detection.

Desirably, the chemical functionality film provided on top of the first surface film is selected from the group consisting of: carboxyl, ether, acrylic acid, acetic acid, acetaldehyde, formaldehyde, amines, ethers, thiols and per-fluoro functionality.

Preferably, the coating comprises a chemical functionality comprises carboxyl.

Suitably, the carboxyl functionality contains the carbonyl group, wherein the carbonyl groups are in the range of from about 0.5 to about 50% and where the content of carbon in said coating is in the range of 1-86 atomic % as determined by XPS. It is preferred that the carboxyl is derived from an acrylic acid precursor.

In one aspect, there is provided a water swellable coating for a solid substrate, the coating comprising:

a first layer comprising siloxane provided on at least one surface of the solid substrate;

a second layer comprising a reactive chemical functionality comprising a carbonyl group, where the content of the carbon in the second layer is in the range of from about 1 to about 86% atomic percent, more preferably the content of the carbon in the second layer is in the range of from about 10 to about 76% atomic percent as determined by XPS, even more preferably the content of the carbon in the second layer is in the range of from about 20 to about 60% atomic percent.

In a preferred embodiment, the content of the carbonyl in the second layer is in the range of from about 2 to about 56% as determined by XPS, more preferably from about 4 to about 36% as determined by XPS, more preferably still from about 5 to about 26% as determined by XPS

The film thickness of the entire layer may vary from about 10 angstroms to about 1000 angstroms, preferably from about 20 angstroms to about 800 angstroms, more preferably from about 25 angstroms to about 600 angstroms, even more preferably from about 30 angstroms to about 400 angstroms, as determined by ellipsometry.

The solid substrate may be any desired 2 or 3-dimensional shape.

Advantageously, the solid substrate coating of the invention swells on contact with water. Advantageously, the substrate coating of the invention has been found to swell by at least a factor of two (2×) when contacted with water. Swelling on the coating may be measured by atomic force microscopy (AFM) by measuring the increase in the root mean square (RMS) roughness of the film in its dry state (in air) and in its wet state (in PBS aqueous buffer). Other techniques that could be used include X-Ray Reflectometry (XRR), quartz microbalance, or ellipsometry.

Further description is given below. Critically, the substrate coating retains strong adhesion with the surface of the substrate despite the large intake of water. It is thought that the non-specific binding of the substrate coating of the invention is reduced by enhancing the interactions of water molecules (hydration sphere) with the coating and also through electrostatic repulsions experienced by negatively charged biomolecules. Thus, the substrate coating of the invention displays high non-specific binding properties, which is a key issue to be addressed in any biomolecule immobilization process.

Desirably, the substrate surface of the invention may comprise a graded layer structure. A graded siloxane network is one which interlinks with the chemical functionality layer. For example, a graded siloxane network is one, which is interlinked with the second layer of functional, reactive chemical functionality, which may be preselected for a particularly desired functionality in a bioassay. A graded structure is one in which the composition varies throughout the thickness of the layers. For example, the substrate coating of the present invention may comprise a graded structure dominated by the siloxane layer or network near the substrate interface that bonds the layer to the substrate, and a highly functionalised highly-charged polymer comprising the plasma polymerised chemical functionality towards the solution interface that can bind biomolecules through formation of covalent bonds. The graded nature of the surface coatings prepared by this method: is important as this means they are silica-rich near the substrate interface which is good for adhesion and washability, while are carbon-rich and highly functionlised near the outer surface for reactivity and non specific binding. The graded layer structure allows layers to be rich in one functionality, for example, carboxylate, amine, ether or thiol functionality at the surface, for example, to allow for further functionalization of the layer and optimum adherence, while filling in pin holes scratches or defects of the substrate surface so that the coating surface is substantially defect free.

The film or coating produced by the method of the invention has been found to adhere well to the substrate surface, so that the substrate may undergo further processing including many wash steps which can occur during biological assay. It is critical that the adhesion of the functional coating to the substrate is as effective as possible, as the system must undergo further processing to perform a complete assay. The PECVD deposited coatings of the invention are subjected to rigorous washing procedures and it was observed that the coating adhesion to COP surface is very good. This is believed to arise from the excellent adhesion to the substrate surface facilitated by the graded nature of the siloxane network layer.

A significant advantage of the present invention is derived from the superior performance of the substrates of the invention over prior art surfaces derived from acrylic acid (AA) or TEOS or HMDSO on their own. A further important advantage is the deposition method of the invention provides, in a industrially repeatable high-throughput manner, the desired bio-device functionality which requires (i) an adherent layer that swells significantly in contact with water, (ii) a graded layer structure dominated by a siloxane network near the substrate interface that bonds the layer to the substrate, and (iii) a highly functionalised highly-charged polymer towards the solution interface, that can bind biomolecules through formation of covalent bond.

Thus, in a related aspect, there is provided for use of a substrate comprising the substrate coating of the invention, comprising a substrate surface having a first layer comprising a siloxane network and a second layer comprising a reactive chemical functionality provided on top of the first surface film to immobilise a target. Thus products comprising the solid substrate having coating of the invention are also contemplated.

Both first and second layers are plasma polymerised layers. The substrate and substrate coating of the invention may be used in an immunoassay. By immunoassay, it is meant any assay which measures the concentration or detects a biochemical substance in a biological liquid, such as serum or urine. Such assays are generally based on the reaction of an antibody to an antigen. Preferably, the immunoassay is an immunosorbent assay, such as an ELISA, a ELISA, a fluorescent enzyme-linked immunosorbent assay, a sandwich enzyme-linked immunosorbent assay, a sandwich fluorescent enzyme-linked immunosorbent assay, a competitive enzyme-linked immunosorbent assay or a direct binding assay. In a related aspect, there is provided the use of a coating for a solid substrate the coating comprising:

a first siloxane layer; and

a second layer comprising a chemical functionality

wherein the first and second layers are plasma polymerized onto the substrate, the coating swellable on contact with an aqueous solution by factor of at least 2, based on RMS (root mean square) roughness swelling measurement to immobilize an analyte in a biodiagnostic application, such as an immunoassay.

In further related aspect there is provided the use of a water swellable coating for a solid substrate the coating comprising:

a first siloxane layer; and

a second layer comprising a chemical functionality

wherein the first and second layers are plasma polymerized onto the substrate, the coating swellable on contact with an aqueous solution by factor of at least 2, based on RMS (root mean square) roughness swelling measurement as a tool for biological discovery and/or biomedical detection, in medical imaging and/or therapeutic applications such as cell labeling, targeted drug delivery, targeted gene delivery, biosensing, cell separation, cell purification and imaging.

Desirably, the substrate and substrate coating of the invention may be used as a tool for biological discovery and/or biomedical detection, in medical imaging and/or therapeutic applications such as cell labelling, targeted drug delivery, targeted gene delivery, biosensing, cell separation, cell purification and imaging. Suitably, the target analyte may be may be a cell, a pathogen, a protein, a molecular label, a molecular tag, a nucleic acid, a detection molecule or a secondary analyte which is highly selective for a further analyte species. More, suitably, the target analyte may be a protein, a detection molecule (capture element) or a secondary analyte which is selective for a further analyte species. Preferably, the detection molecule and/or the secondary analyte is an antibody. It will be appreciated that an antibody that recognizes the target analyte is called the “primary antibody”. Suitably, the capture element is Cy5-labeled oligonucleotide or Cy5-labeled goat anti-human IgG.

Suitably, the immunosorbent assay is a sandwich assay. This is desirable to since it is one of the most powerful immunosorbent assay formats. This is a type of capture assay which is called a “sandwich” assay because the target analyte to be measured is bound between two primary antibodies, i.e., the capture antibody which is used to immobilize the target analyte onto a support and the label of the invention which comprises a detection antibody which is selective for the target analyte. The sandwich format is preferred since it is a sensitive and robust technique. This type of method involves an indirect immunosorbent assay procedure in which the analyte is detected, by actual detection of a secondary analyte, which is probed for, by the detection molecule of the label of the invention. The key step of the assay is the immobilization of the target analyte to the support phase (for example, an antigen). Desirably, immobilization of the target analyte can be accomplished by direct adsorption to the assay plate. Alternatively, the target analyte may be immobilized onto the support phase indirectly through use of a capture molecule (for example, an antibody) that has been attached to the plate before it is exposed to the target analyte.

There is also provided a kit for testing for target analyte, the kit comprising:

(i) substrate, comprising the substrate coating of the invention;

(ii) optionally, capture molecule;

(iii) optionally, detection molecule; and

(iv) instructions for use.

The kit of the invention may further comprise activating reagents. Desirably, the kit may further comprise buffer and/or wash solutions.

### BRIEF DESCRIPTION OF THE DRAWINGS

The invention will be more clearly understood from the following description of the invention, given by way of example only, with reference to the accompanying drawings, in which:

FIG. 1: A—Schematic showing the concept of functionalizing a substrate surface using siloxane precursors with built-in functional groups (left) or co-deposition of simple siloxane and other volatile precursor that includes the desired functional group; B—schematic showing specific and non-specific binding (NSB);

FIG. 2: A—Thickness of selected coatings of the invention deposited by PECVD; B—uniformity of spots deposited as 1 mL drops on the APTES surface;

FIG. 3: MS analysis of fragmentation of AA and TEOS precursors in argon as a function of increased plasma power;

FIG. 4: A—Specific and non-specific binding of Cy5-labeled oligonucleotide; B—Specific and non-specific binding of Cy5-labeled goat anti-human IgG;

FIG. 5: Atomic force microscopy images of coated surfaces of the invention prepared by PECVD; swelling of the coatings are measured by atomic force microscopy (AFM) by measuring the increase in the root mean square (RMS) roughness of the film in its dry state (in air) and in its wet state (in PBS aqueous buffer);

FIG. 6: Interactions of dye doped nanoparticles with surfaces prepared by PECVD;

FIG. 7: Varying interfacial tension on PECVD prepared surfaces measured as changes in water contact angles;

FIG. 8: Patterned surface on COC slide;

FIG. 9: ATR-FTIR of plasma deposited coatings showing the presence of carboxy functionality in TA and TAP slides and the absence of it in uncoated Zeonor;

FIG. 10: Schematic illustration of assessment method to evaluate specific vs. non-specific binding capabilities of the surfaces of the invention;

FIG. 11: A chart showing the specific vs. non-specific binding properties of studied surface s comprising acrylic acid chemical functionality over TEOS and HMDSO siloxane network;

FIG. 12: Contact angle comparison showing the ageing effect of plasma deposited carboxy functionality by PECVD;

FIG. 13: A concept showing the relationship between the chemical composition of the surface measured by XPS and the surface capacity for specific and non-specific binding of DNA and model IgG antibody;

FIG. 14: Assay curves illustrating the effect of PECVD prepared surfaces based on co-deposition of TEOS, acrylic acid and PEG derivative (AA/PEG in the left plot) on the limit of detection and assay sensitivity. The PECVD film was compared with PDITC film that can be found in many existing commercial devices;

FIG. 15: DNA hybridization assays Ψspectra measurements by total internal reflection ellipsometry (TIRE);

FIG. 16: hCG sandwich immunoassays Ψspectra measurements by total internal reflection ellipsometry (TIRE);

FIG. 17: Secondary ion mass spectroscopic spectra of TEOS coating on gold. (Top) spectra taken at the surface (middle) Corresponds to an etched layer by gallium ion in the SIMS and (Bottom) corresponds to deeper layer close to gold interface; and

FIG. 18: Spectroscopic ellipsometry measurement of Teos coated on cold surface and the film thickness measured before and after exposing to PBS Tween buffer.

Table 1: XPS analysis of selected films prepared by co-deposition of TEOS(HMDSO) and acrylic acid at different conditions; and

### DETAILED DESCRIPTION OF THE INVENTION

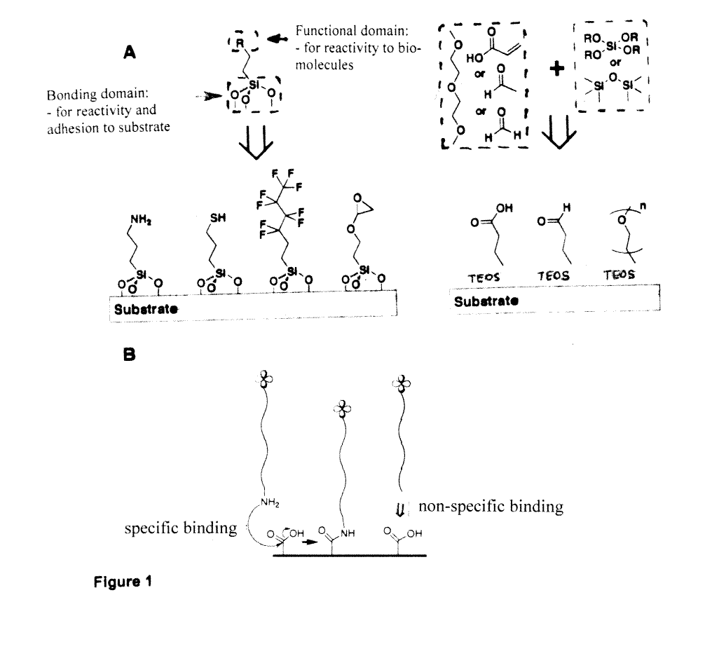

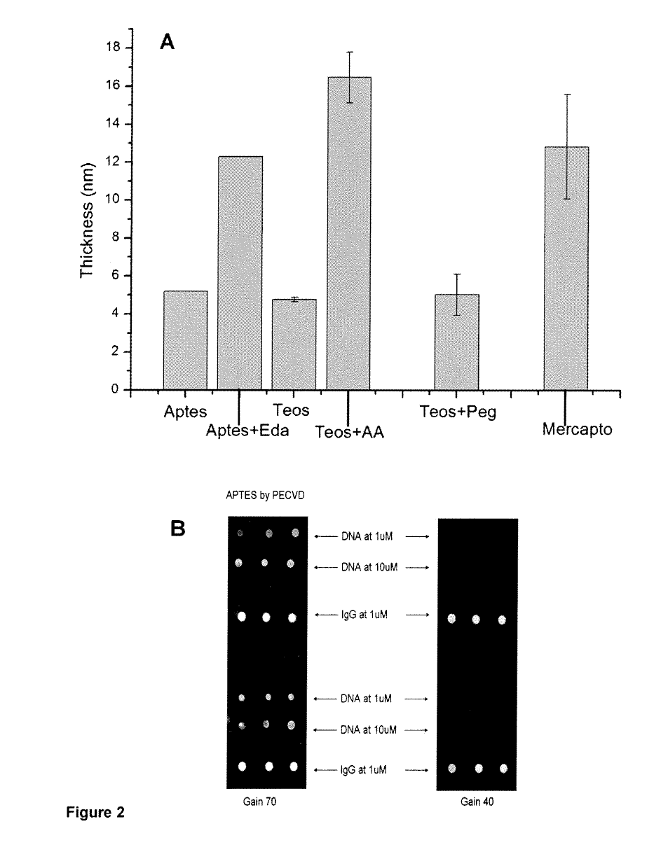

The Inventors have now provided a process for the chemical functionalization of a substrate surface, exemplified herein by a cyclic olefin polymer (COC) (known as Zeonor). The method uses plasma enhanced chemical vapour deposition (PECVD) and produces surface coating that have many favourable properties and in particular are useful in bioanalytical techniques (FIG. 1) as the substrate comprising the coating of the invention:

(i) possess a high degree of functionality for binding of biorecognition elements;

(ii) facilitate retention of the specific binding activity of the target to a high degree; and

(iii) minimize the general non-specific binding (NSB) of non-analyte constituents of the sample. In general, such the coatings of the invention are hydrophilic and exhibit high stability, high degree of swelling and high specific-to-non-specific binding ratio.

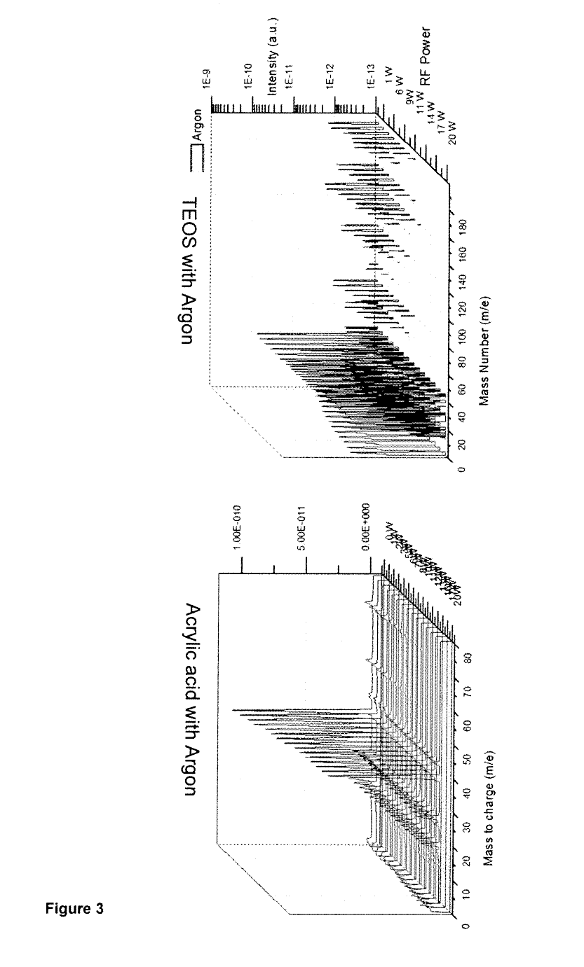

Precise control of the plasma parameters is needed to obtain the required substrate chemical functionality. It has been found that the power setting in the PECVD process affects the fragmentation of the chemical functionality precursors, and this has been found to have a significant effect on the functionality and properties of the final surface. The Inventors have identified a range of plasma power conditions, at which the precursor fragmentation is optimal and the substrate coatings of the invention show high stability and functionality. It will be appreciated that plasma power can be rather dependent on the specific characteristics of any particular plasma apparatus. Therefore, the Inventors have used a mass-spectrometric method screening step on the plasma in order to identify the instrument conditions needed to produce the power necessary to achieve optimum fragmentation of the precursors. This is illustrated in FIG. 3 below for precursors acrylic acid and TEOS in an argon plasma. A desirable precursor fragmentation pattern is indicated by an MS precursor fragmentation pattern in which the plasma power is sufficiently high enough to activate the surface of the solid substrate by providing reactive oxygen and or hydroxyl containing species on the surface, but is sufficiently low enough to avoid significant fragmentation of the chemical functional groups deposited in step (iii).

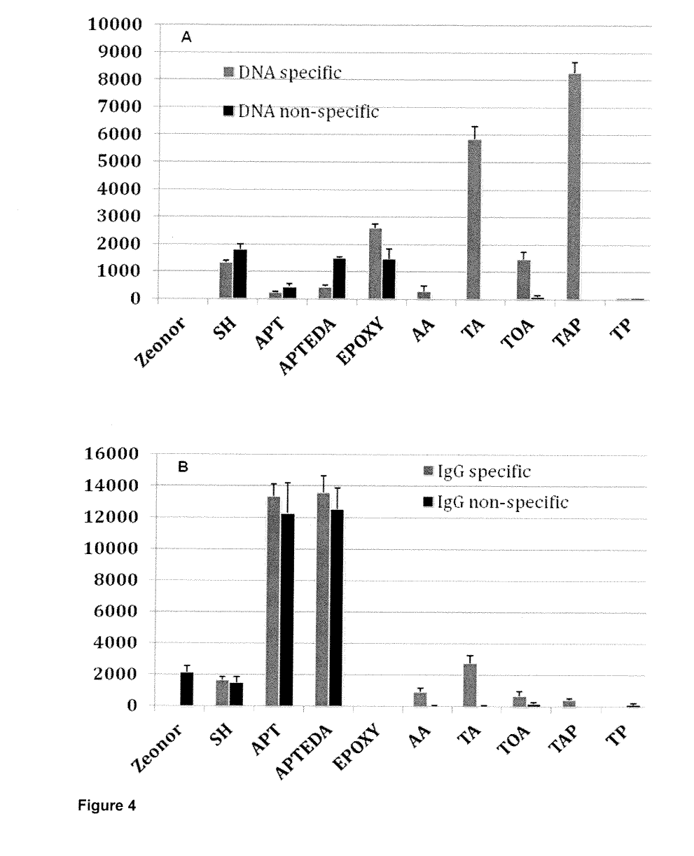

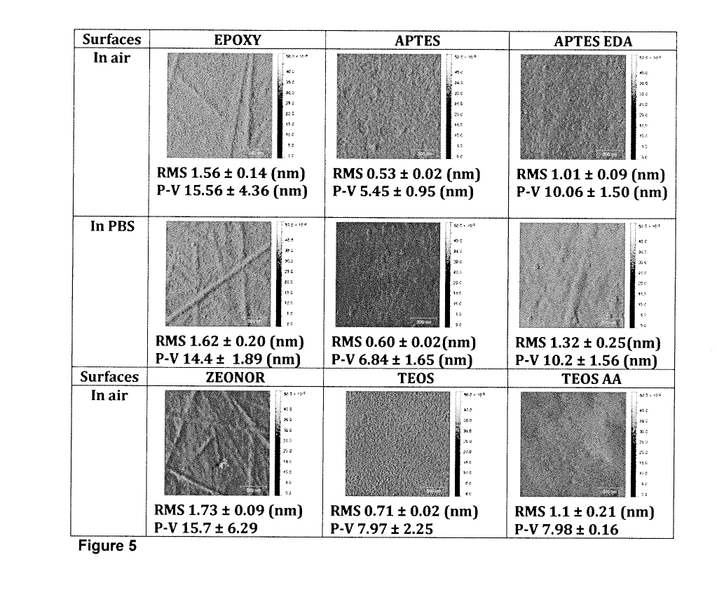

The chemical and physical characterization of the plasma deposited films or coatings was performed using a multi-technique approach including X-ray photoelectron spectroscopy (XPS), X-ray reflectivity (XRR), attenuated total reflectance Fourier transform infrared spectroscopy (ATR-FTIR), fluorescence microscopy (after attaching reactive fluorophore), contact angle analysis and atomic force microscopy (AFM) analysis. In particular, the desirable non-specific binding properties discussed above were measured by fluorescence based techniques by measuring the signal intensity of the capture-elements (proteins or nucleic acids) on the coated substrate surface (hence called SPECIFIC binding) to the signal intensity of the detection-elements at zero concentration of analyte (NON-SPECIFIC binding). The results are summarized in FIG. 4A & FIG. 4B. This experiment demonstrates that the plastic COC surfaces prepared by co-deposition process of siloxane (TEOS or HMDSO) and functional monomers (AA, PEG etc.) show superior characteristics in terms of binding capacity and reduction of non-specific binding when compared to the alkylsilane precursors.

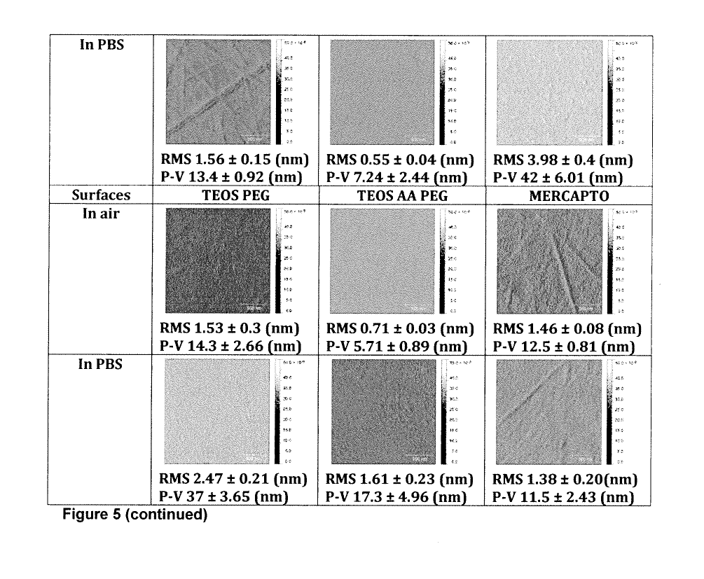

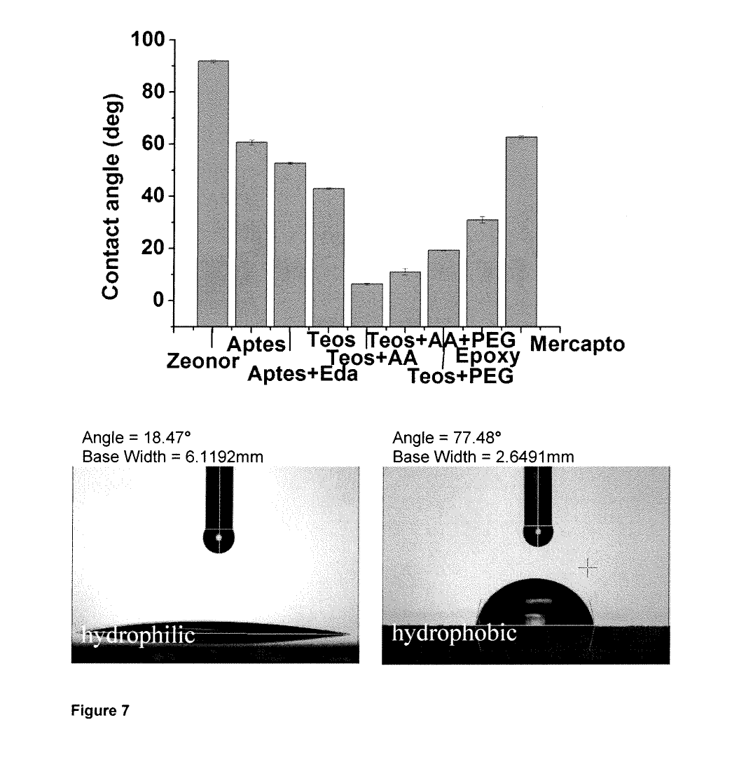

The Inventors' findings indicate that one key characteristic of the substrate coating of the invention having significant influence on the way the biomolecule is immobilized onto the coated surface is the degree of coating swelling upon contact with water. An ideal surface should have soft-like character and should swell when exposed to molecules of water. Extensive research on the amount of non specific binding and the water absorption characteristics of the surface coatings of the present invention and in particular coatings comprising carboxy chemical functionality deposited by PECVD. The swelling properties of the PECVD prepared coatings of the invention have been investigated using atomic force microscopy (AFM). The changes in surface roughness was measured in dry and wet (immersed in PBS buffer) modes using atomic force microscopy (AFM). The results are summarized in FIG. 5. As seen on FIG. 5, the pristine COC surface (Zeonor) exhibits relatively large roughness, characterized by scratches and pinholes. However, plasma deposition of a TEOS layer improves the root mean square (RMS) factor and makes the surface smoother by filling up cracks, pinholes and other defects that were on the original pristine substrate surface. After the TEOS layer is deposited, the acrylic acid is deposited in the same chamber sequentially. It is believed that the TEOS molecules initially form a thin bonding layer onto the COC substrate surface, onto which acrylic acid (AA) is further polymerized. The polymerization process is initialized and assisted by the plasma and results in a fabrication of a relatively homogeneous layer with high density of carboxylic acid functionality arising from use of acrylic acid. Swelling of the TEOS-AA layer is characterised by the increase in the root mean square (RMS) roughness of the film in its dry state (in air) and in its wet state (in PBS aqueous buffer) as determined by AFM. The very low water contact angle of the film (see FIG. 7) also suggests that TEOS may be inserted into the sensing COOH layer, cross-linking the polymerized acrylic acid molecules and forming an abundance of silanols and silyl ethers. The acrylic acids also contribute to the significant swelling of the film upon contact with water as seen in the increase of the RMS and P-V roughness of the films in FIG. 5.

In fluorescent linked immunoassays, dye doped nanoparticles (NPs) can be used to significantly enhance the signal. However, the benefits of using such bright labels could be only realized if the background response (non-specific binding) is low enough, which of course is achieved by the present substrate coatings.

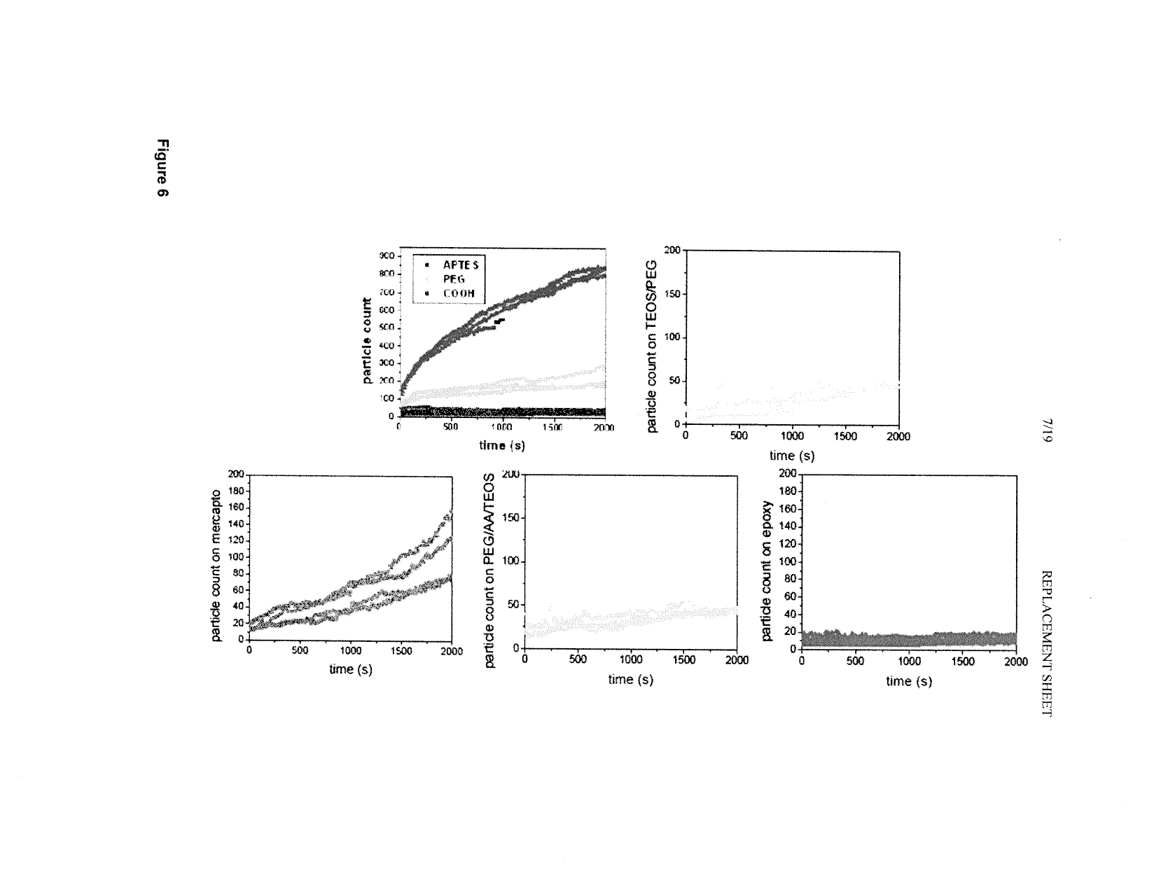

Furthermore, the Inventors have sensitively measured the non-specific binding (NSB) of antibody-sensitized NPs in the absence of antigen on substrate surfaces prepared by the PECVD method of the present invention. TIRF microscopy with image capture and processing was used to measure directly the collision frequency of NP labelled particles with the capture surface and their residence time as seen in FIG. 6. All substrate coatings of the invention having —COOH chemical functionality showed lower degree of non-specific binding than surfaces where PEG was used, which suggests that the physisorbtion of the studied NPs is mostly controlled by electrostatic repulsions between the negatively charged —COOH surface and negatively charged nanoparticles.

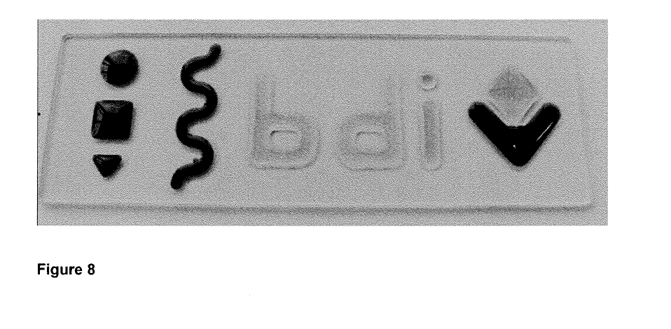

Another important requirement for specific to single-use bioanalytical devices that rely on capillary forces, is the ability to tailor the interfacial tension of the surface in order to control fluid flow in the device. Interfacial tension tailorability is demonstrated by changes of the water contact angle on the PECVD deposited substrate coatings of the invention. Thus, the Inventors have developed a tool that allows the preparation of surface coatings with a tailorable broad range of interfacial tension (from hydrophilic to hydrophobic) as seen in FIG. 7. Each of these surface substrate coatings will have varying fluid flow properties. In some cases, it is desirable to be able to pattern the interfacial tension, which is achievable by either masking the substrates with plasma resistant tape or with the use of excimer laser to ablate the coating through to the hydrophobic substrate thus creating a hydrophobic barrier to spreading of liquid on the surface (FIG. 8). This will facilitate chip design.

From the data presented herein, the substrate surface coatings of the invention prepared by co-deposition of siloxane derivative (TEOS, HMDSO etc.) and functional monomers (AA, AcA, AH etc.) show superior characteristics to surfaces comprising their alkylsiloxane derivatives (APTES, GOPTMS, MPTES etc.).

### TEOS—Acrylic Acid Films (and Similar)

Significant fragmentation of the precursor feed generally occurs in glow discharges and as a result, a wide range of functional groups may appear in the coating. The fragmentation can be minimized by carefully tuning parameters such as power input and duty cycle to obtain a high degree of monomer structure retention in the film. As discussed above for each precursor, the optimum plasma power for a particularly plasma machine can be determined by mass spectral analysis of the precursor in plasma.

In the work presented in this section, the surfaces of COC coated substrates were modified by the methods of the invention to functionalise them with carboxylic acid functionality. Such coated substrates are useful in applications involving immunoassay devices capable of immunoassay. Thus, the focus of this section is on an investigation into tetraethylorthosilicate (TEOS) and acrylic acid (AA) as precursor sources to produce the hydrophilic substrate coatings of the invention having carboxy functionality. The substrate surface coating of the invention was characterised by contact angle measurement to monitor changes in wettability and ageing effects, fluorescence microscopy to study the reactivity and non specific binding of the plasma deposited functionality, attenuated total reflection-Fourier transform infra red (ATR-FTIR) spectroscopy to study the presence and amounts of carboxy functionality, XPS to study the chemical composition, AFM for nanoscale topography and TIRE to investigate the thickness of the coatings.

The chemical composition of the surface coating of invention has been found to be dependant on several variable method parameters applied during the deposition process. For example, the coating thickness, contact angle, elasticity, swelling properties and the density of functional groups can be controlled by varying the plasma deposition times and chemical nature of the precursors. TEOS and acrylic acid (AA) have been chosen as model precursors for forming a typical substrate coating of the invention. A relatively wide range of samples has been prepared by changing the plasma deposition time of TEOS and AA and measuring the quantitative differences in chemical composition of all samples by X-ray photoelectron spectroscopy (XPS) and qualitatively by ATR-FTIR.

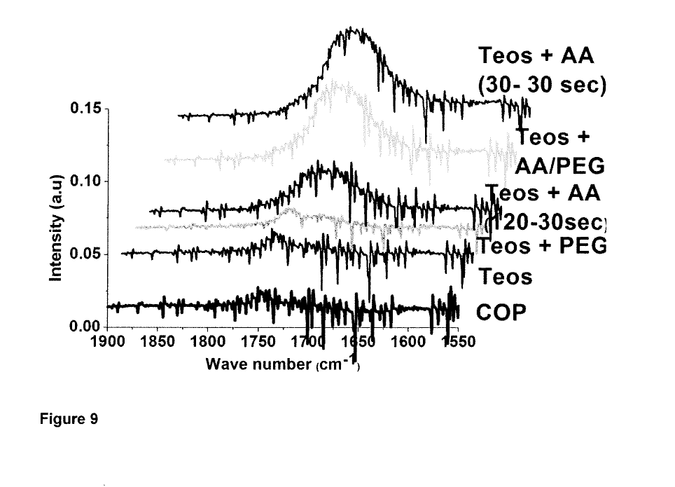

ATR-FTIR spectra of FIG. 9 shows the carboxylic acid C═O stretching peak for the plasma deposited coatings comprising TEOS and AA. The CO═O peak intensity of TEOS+AA and TEOS+AA/PEG coating shows a significantly higher intensity compared to that of TEOS and TEOS+PEG coating. The presence of very low quantity of carboxy functionality in TEOS coating was observed in XPS spectra (Table 1). The plasma polymerisation of TEOS results in formation of a very low quantity of carboxy functionality (5.92%) due to the presence of residual oxygen content in the chamber after the pre-treatment process and also from the oxygen content in the TEOS precursor. However, the addition of acrylic acid in the plasma process resulted in a significant increase in carboxy functionality (24.69%) with 2 minutes of carboxy deposition. The amount of carboxy functionality was further increased by using HMDSO as siloxane precursor as the adhesion and network building layer and acrylic acid as the precursor source of carboxy functionality. With a sequential deposition of HMDSO and AA for 30 seconds each, the carboxy functionality was increased to 25.98%. Acrylic acid deposited on its own without the use of adhesion and network building siloxane layer resulted in 19.66% of carboxy functionality. However, the biomolecule attachment carried out using Cy5-labeled oligonucleotide and Cy5-labeled goat anti-human IgG, in FIG. 4 showed that the signal intensity was very low in AA coating compared to that of TEOS+AA coating. It is evident from FIG. 4 that the presence of siloxane layer increases the signal intensity significantly and quantitative chemical composition measurement by XPS (Table 1) confirms the presence of significantly higher carboxy functionality on siloxane (HMDSO or TEOS) and acrylic acid combination.

TABLE 1
XPS analysis of selected films prepared by co-deposition of TEOS (HMDSO) and
acrylic acid at different plasma conditions

| Sequential | | Binding energy (eV) | | | | | |
|---|---|---|---|---|---|---|---|
| deposition of | | (% Area of Peak in C1s) | | | At % in the sample | | |
| | AA (sec) | C—C | C—O | C═O | C | O | Si |
| Teos + (sec) | | | | | | | |
| 120 | 0 | 285.0 | 286.3 | 289.3 | 21.20 | 64.79 | 14.01 |
| | | (66.80%) | (27.20%) | ( 5.92%) | | | |
| 30 | 30 | 285.0 | 286.1 | 289.1 | 49.27 | 48.15 | 2.58 |
| | | (65.92%) | (21.44%) | (12.65%) | | | |
| 120 | 5 | 285.0 | 286.0 | 289.1 | 31.24 | 57.35 | 11.41 |
| | | (52.94%) | (30.57%) | (16.49%) | | | |
| 120 | 120 | 285.0 | 286.0 | 289.0 | 40.51 | 58.13 | 1.35 |
| | | (50.23%) | (25.08%) | (24.69%) | | | |
| 0 | 120 | 285.0 | 286.2 | 289.1 | 51.67 | 48.01 | 0.31 |
| | | (63.33%) | (17.01%) | (19.66%) | | | |
| Teos/O₂ + (sec) | | | | | | | |
| 120 | 120 | 285.0 | 286.1 | 289.1 | 32.69 | 58.64 | 8.68 |
| | | (67.81%) | (20.31%) | (11.87%) | | | |
| HMDSO/O₂ + (sec) | | | | | | | |
| 30 | 30 | 285.0 | 285.9 | 289.0 | 59.23 | 35.92 | 4.85 |
| | | (47.50%) | (26.53%) | (25.98%) | | | |

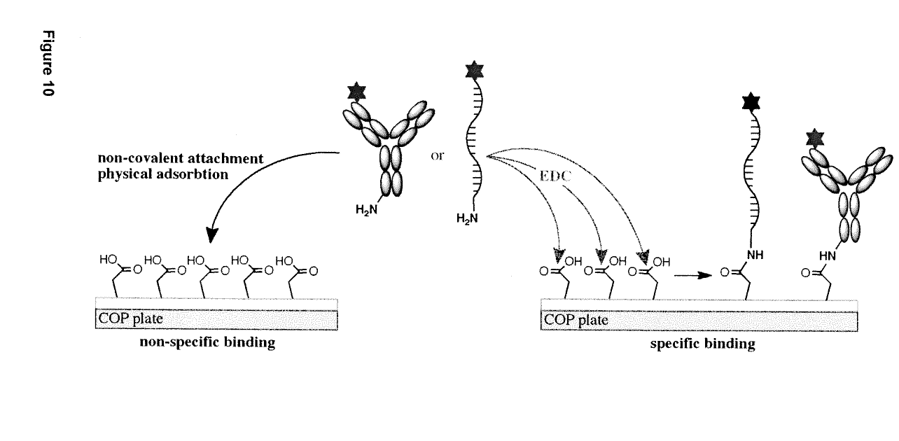

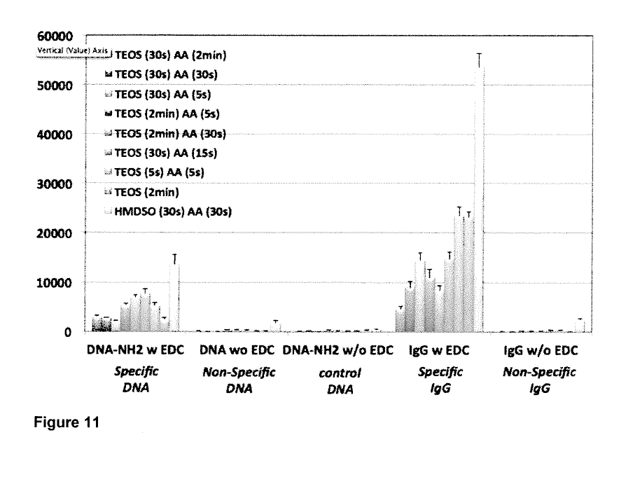

Furthermore, the Inventors have probed the surface binding capacity and the effect of non-specific binding on all of the model TEOS/AA substrate surface coatings by reactions with Cy5-labeled oligonucleotide and Cy5-labeled IgG. The surface loading capacity was measured by allowing the surface —COOH groups to react with free amines (—NH₂) present in proteins and also in amino-modified oligonucleotide. For the covalent attachment, the —COOH groups were activated by dehydrating agent such as 1-ethyl-3-[3-dimethylaminopropyl]carbodiimide hydrochloride (EDC). In the presence of EDC, the protein (as well as the amino-modified DNA) form a stable covalent amide bond on the surface. To assess the degree of non-specific binding, fluorescent labelled protein or DNA molecules were adsorbed on the surface at zero concentration of the analyte and in the absence of EDC, as illustrated on FIG. 10. The results of the experiment described above are summarized in FIG. 11. Interestingly, shorter deposition times of AA (15 s and 30 s being the best) lead into formation of films with higher binding capacity and lower non-specific binding. It is expected that the presence of highly charged ionic and radical species in plasma initiates the polymerization of the acrylic acid and facilitates its crosslinking with the underlying TEOS adhesion and network building layer. At longer exposure to such reactive species in plasma, it is thought that fragmentation may occur, which could lead into formation of loosely bound, volatile fragments on the film that are subsequently washed away during the biomolecule-immobilization and related washing steps.

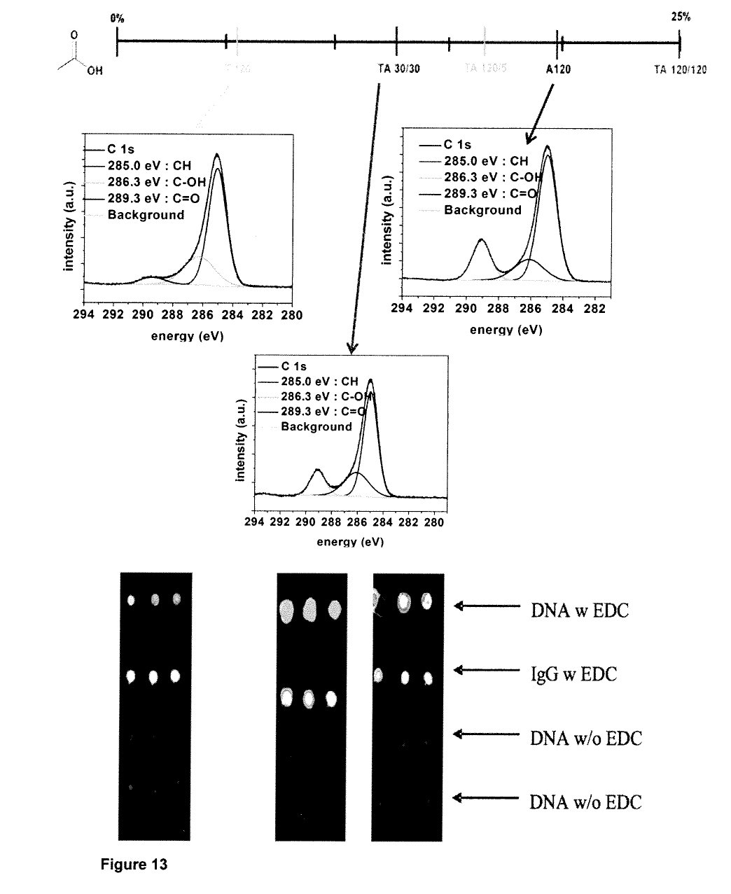

The aim of both the XPS and the fluorescence studies was determine a relationship between the chemical composition of the surface and its performance in terms of binding capacity and reduction of non-specific is binding. The basic illustration of such concept is shown on FIG. 13.

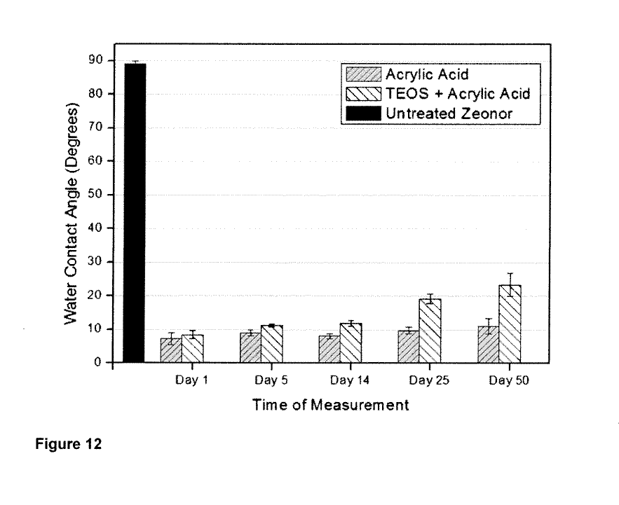

Furthermore, the Inventors have performed an ageing study of the TEOS/AA substrate coating stored in air for over 50 days. The stability of the coating was measured as a function of changing contact angle parameters (FIG. 12). Also, FIG. 12 illustrates excellent adhesion properties of films prepared by sequential deposition of TEOS+AA according to the method of the invention. Advantageously, the TEOS+AA films of the present invention retain their hydrophilicity even when stored on air for more than 50 days. The increase in contact angle to −20 degrees is attributed to the presence of contaminants in water and in air.

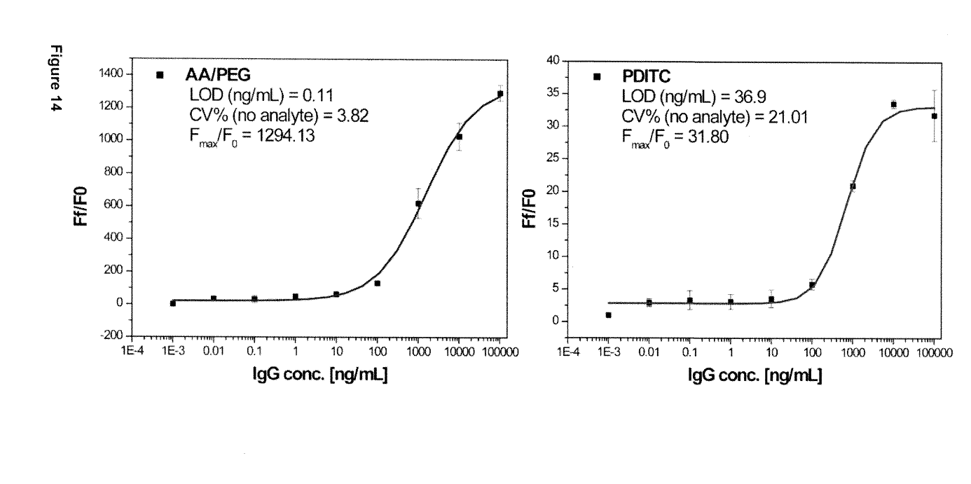

FIG. 14 illustrates how a model immunosorbent assay was performed to illustrate how reduction in background response translates into improvements in signal-to-noise ratio and subsequently to improved limit of detection. A carboxylic-functionalized film in a matrix of protein-resistant PEG was fabricated on cyclo olefin polymer substrate by PECVD. Such a substrate coating in its highly reactive form enabled covalent attachment of biomolecules on the surface, while its low reactivity form prevented the unspecific binding of other non-analyte solution constituents. The surface capacity of suppressing background fluorescence was demonstrated on a prototype of biochip device based on supercritical angle fluorescence detection. The sensitivity improved by 2 orders of magnitude as a direct consequence of the optimal surface chemistry (FIG. 14). The effect of coating with incorporated PEG matrix was compared to other films prepared by either wet chemistry methods or plasma assisted vapor technique. The results indicate that the PECVD process of the invention is indeed capable of preparing surfaces, characteristics of which are comparable or even better to those that are frequently used in immunoassays by other scientific and industrial entities.

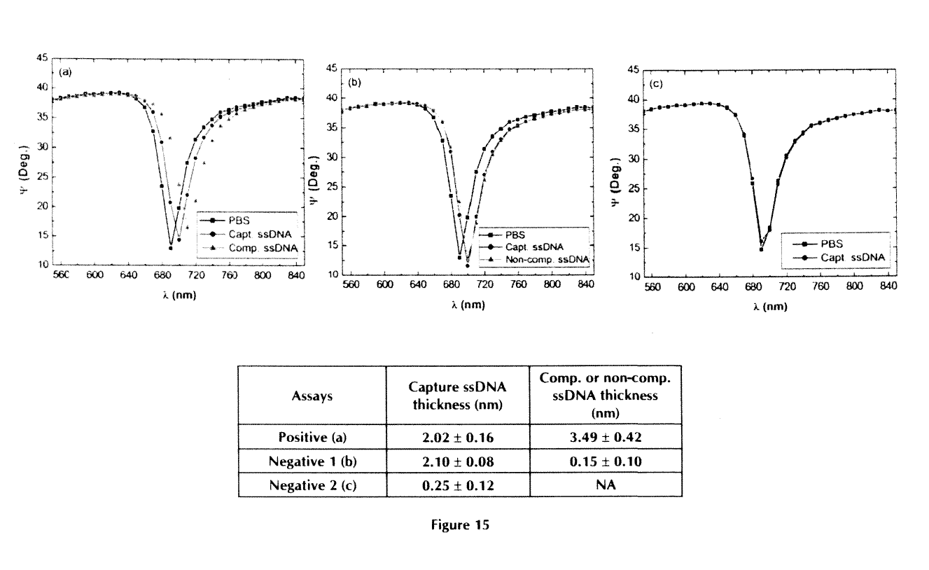

The TEOS/AA surface coatings of the invention have also been shown to have high binding capacity when used in DNA hybridization as well as sandwich hCG immunoassays. The inventors have used surface plasmon resonance-enhanced total internal reflection ellipsometry to assess the efficiency of surface binding. The Ψspectra measured by this technique are sensitive to surface binding changes. The Ψspectra of the complete DNA hybridization assays are plotted in FIG. 15, respectively. The introduction of the capture aminated Sa19 ssDNA (15-mer) solution and then the complementary Sa19 rev comp ssDNA (15-mer) solution resulted in large shifts in Ψ spectra from the initial Ψ spectra of the COOH surface when the microwell was filled with PBS buffer (FIG. 15(a)). In the first negative control experiment, a mismatched Sa20 non-comp ssDNA solution (20-mer) was incubated for 60 min after the capture Sa19 ssDNA had been immobilized to assess the non-specific hybridization and non-specific binding effect. As seen in FIG. 15(b), the introduction of the non-complementary ssDNA solution did not result in any shifts in the Ψspectra. Therefore, the effects of non-specific binding and non-specific hybridization were minimal. The second negative control experiment, conducted by incubating the aminated Sa19 ssDNA solution without the activating agent EDC, showed that only a very small shift in Ψspectra were observed after 60 min (FIG. 15(c)). The results of the fitting of the thicknesses of the ssDNA in three assays were summarized in the table.

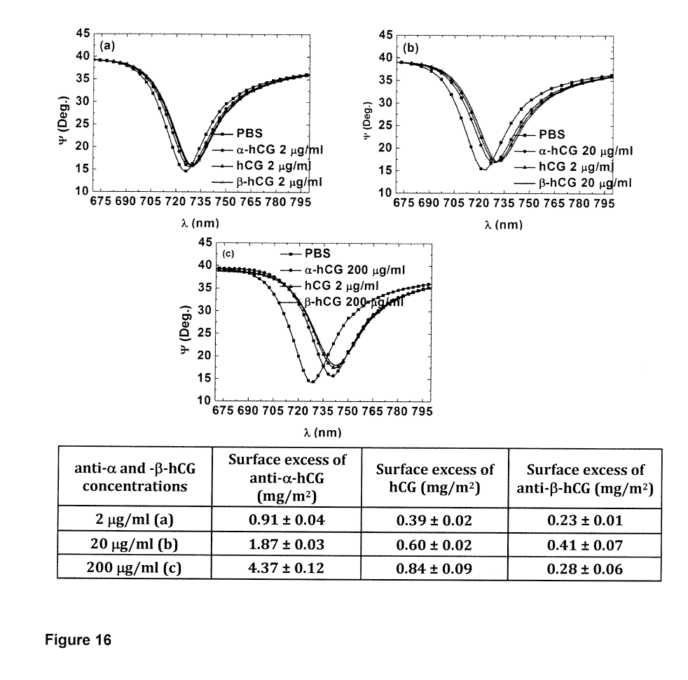

Similarly to the DNA hybridization assays, the hCG sandwich immunoassays were successfully realised after activating the COOH surface with EDC/NHS mixture inside the TIRE microwells. The covalent binding of the capture anti-α-hCG antibody caused a large and distinct shift in Ψspectra. These shifts increased with increasing concentration of the anti-α-hCG, in the range from 2 to 200 μg/ml (FIG. 16(a-c)). The subsequent binding of the hCG antigen at 2 μg/ml to the capture anti-α-hCG caused another shift in Ψspectra (FIG. 16(a-c)). Finally, the binding of the second antibody anti-β-hCG caused a third shift in Ψspectra (FIG. 16(a-c)). The results of the fitting and the calculation of the protein surface excesses for three hCG immunoassays were summarized in the table. The surface excesses of the anti-α-hCG antibodies increased with increasing concentrations of the capture anti-α-hCG in the initial surface functionalization.

Overall, functional coatings have been successfully deposited by to plasma enhanced chemical vapour deposition on substrate substrates using siloxane as an adhesion and network building layer and have been found to result in low non specific binding, high signal to noise ratio and improved limit of detection, particularly in the case of —COOH surface functionality. X-ray photoelectron spectroscopic measurement showed the presence of COOH. The presence of siloxane functionality, that was essential for film adhesion to COP, was also confirmed. The fluorescent scans confirmed the reactivity of the amine terminated DNA with the carboxyl groups of the surface. Despite the contact angle being larger than that of just Acrylic Acid coatings the TEOS+FAA still showed considerable change in contact angle from native COP. The increased signal to noise ratio (FIG. 3 Right) showed great improvement and the possibility for earlier detection of disease markers in biodevices.

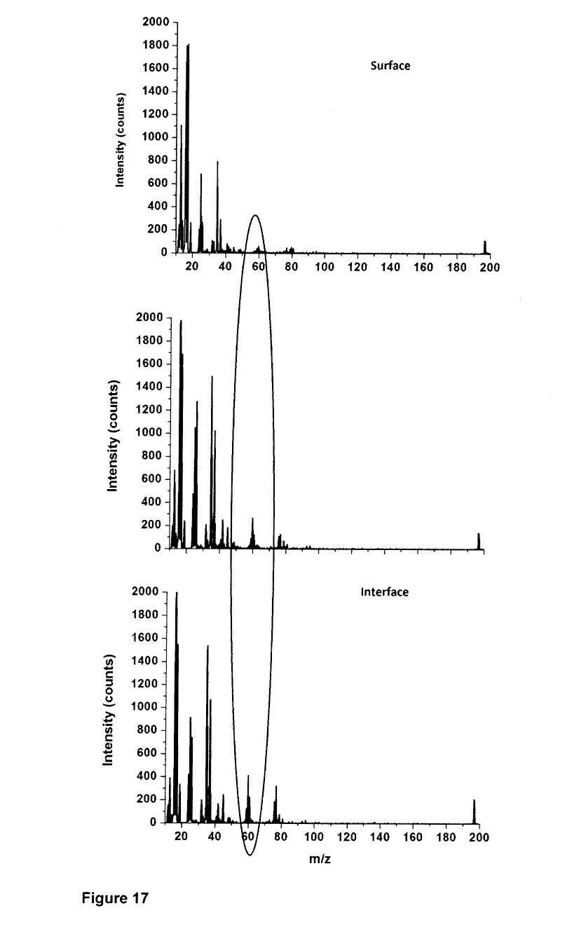

The plasma functionalisation of carboxylic groups using siloxane as a network building layer is not only limited to plastic substrates including cyclo olefin copolymer, polycarbonates and PMMA but also to metallic surfaces including gold and silver. PECVD of TEOS on gold layer showed the presence of siloxane layer at the interface. FIG. 17, shows the surface—interface analysis of the TEOS coating on gold substrate using secondary ion mass spectroscopy (SIMS). The SIMS spectra shows three different scans taken at the some location corresponding to various depths in the coating.

Mass/Charge ratio (m/z) 12 corresponds to Carbon and 60 corresponds to SiO₂. It is evident that the top layer is rich in carbon as the m/z 12 signal is close to 1100 counts and the SiO₂ signal is low, close to 50 counts. Deeper in the surface, the carbon content decreases and the SiO₂ signal increases. The bottom figure shows that the carbon intensity decreased to 400 counts from 1100 counts at the surface and the SiO₂ intensity increased from 50 counts at the surface to 400 counts at the interface. This illustrates what is believed to be an important characteristic of the surface coatings prepared by this method: that they are graded in composition, being silica-rich near the substrate interface and carbon-rich and highly functionlised near the outer surface. Grading of the layer composition is achieved as consequence of ion-induced mixing of the surface coating, under the application of the plasma. A higher SiO₂ content and low carbon content at the interface could possibly be explained by two reasons (i) the oxygen plasma pretreatment of the gold surface results in higher oxygen content at the surface which then binds to Si in TEOS resulting in high SiO₂ content at the interface and (ii) the presence of residual oxygen content in the chamber due to plasma pretreatment results in oxidation of the TEOS precursor resulting in formation of SiO₂ at the surface and CO₂ and H₂O in the bulk plasma that are pumped out of the chamber. As the deposition continues, the residual oxygen is used up by the TEOS precursor and the oxygen in TEOS is not sufficient to fully oxidise the precursor resulting in lower oxidation of the precursor and hence a higher carbon content and low SiO₂ content in the film. This demonstrates that the gold surface could also be functionalised using the same procedure as that of cyclo olefin copolymer.

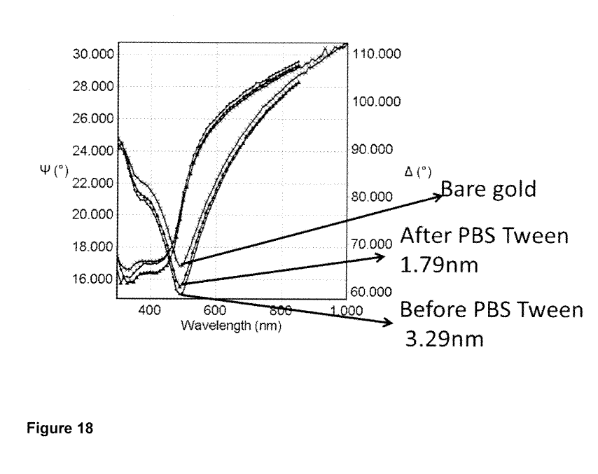

The spectroscopic ellipsometry measurement (FIG. 18) carried out on TEOS coated gold surface demonstrates that the TEOS coating adheres well to gold following oxygen+argon plasma activation and the coating stays after rigorous washing with PBS Tween. A decrease in film thickness after washing from 3.29 nm to 1.79 nm is probably because of the removal of loosely bound particles on the surface. From the spectroscopic ellipsometry and SIMS measurement it is evident that the siloxane (TEOS or HMDSO) could be use as an adhesion and network building layer for functionalisation on both plastics and on gold.

### REFERENCES

1. H. Becker and C. Gartner, Analytical and Bioanalytical Chemistry, 2008, 390, 89-111.
2. C. Jonsson, M. Aronsson, G. Rundstrom, C. Pettersson, I. Mendel-Hartvig, J. Bakker, E. Martinsson, B. Liedberg, B. MacCraith, O. Ohman and J. Melin, Lab on a Chip, 2008, 8, 1191-1197.
3. S. A. Kanan, W. T. Y. Tze and C. P. Tripp, Langmuir, 2002, 18, 6623-6627.
4. C. Voulgaris, A. Panou, E. Amanatides and D. Mataras, Surface & Coatings Technology, 2005, 200, 351-354.
5. C. Volcke, R. P. Gandhiraman, V. Gubala, J. Raj, T. Cummins, G. Fonder, R. I. Nooney, Z. Mekhalif, G. Herzog, S. Daniels, D. W. M. Arrigan, A. A. Cafolla and D. E. Williams, Biosensors & Bioelectronics, 2010, 25, 1875-1880.
6. F. Rusmini, Z. Y. Zhong and J. Feijen, Biomacromolecules, 2007, 8, 1775-1789.
7. L. Detomaso, R. Gristina, R. d'Agostino, G. S. Senesi and P. Favia, Surface & Coatings Technology, 2005, 200, 1022-1025.
8. L. Detomaso, R. Gristina, G. S. Senesi, R. d'Agostino and P. Favia, Biomaterials, 2005, 26, 3831-3841.
9. X. Z. Cao, T. Z. Zhang, Q. T. Nguyen, Y. Y. Zhang and Z. H. Ping, Journal of Membrane Science, 2008, 312, 15-22.
10. R. P. Gandhiraman, C. Volcke, V. Gubala, C. Doyle, L. Basabe-Desmonts, C. Dotzler, M. F. Toney, M. Iacono, R. I. Nooney, S. Daniels, B. James and D. E. Williams, Journal of Materials Chemistry, 2010, 20, 4116-4127.
11. H. Jiang, K. Eyink, J. T. Grant, J. Enlow, S. Tullis and T. J. Bunning, Chemical Vapor Deposition, 2008, 14, 286-291.
12. T. Fujii, M. Hiramatsu and M. Nawata, Thin Solid Films, 1999, 343, 457-460.

1. A method of preparing a solid substrate coating that swells on contact with an aqueous solution by factor of at least 2, based on a RMS (root mean square) roughness swelling measurement, the method comprising the steps of:
(iv) activating a surface of the solid substrate by treatment with a plasma;
(v) depositing a first layer of siloxane onto the surface of the solid substrate using plasma; and
(vi) depositing a second layer of at least one chemical functionality on top of the first layer using plasma.

2. The method according to claim 1 wherein the plasma arises from a plasma enhanced chemical vapour deposition technique (PECVD).

3. The method of claim 1 wherein the siloxane is deposited by plasma polymerisation of a siloxane precursor chemical compound and the at least one chemical functionality is deposited by plasma polymerisation of at least one chemical functionality precursor chemical compound.

4. The method of claim 1 wherein the plasma power is selected to activate the surface of the solid substrate by providing reactive oxygen and or hydroxyl containing species on the surface and to supply sufficient energy to promote the chemical reactions that form an adherent polymerized surface layer but without excessive fragmentation of the chemical functionality deposited in step (iii).

5. The method of claim 1 wherein the plasma power necessary is determined by a mass-spectrometric analysis of at least one precursor in the plasma.

6. The method of claim 4 wherein the plasma power is determined by adjusting the plasma parameters to produce a mass spectroscopic fragmentation pattern corresponding to one of patterns of siloxane precursor or a chemical functionality precursor as shown in FIG. 3.

7. The method of claim 1 wherein steps and (iii) are carried out simultaneously in a co-deposition step.

8. The method of claim 1 wherein step (i) utilises a plasma Radio Frequency (RF) power in the range of from about 20 Watts to about 300 Watts for a period of from about 30 seconds to about 5 minutes.

9. The method of claim 1 wherein step (ii) utilises a plasma Radio Frequency (RF) power in the range of from about 5 Watts to about 300 Watts for a period of from about 5 seconds to about 30 minutes.

10. The method of claim 1 wherein step (iii) utilises a plasma Radio Frequency (RF) power in the range of from about 5 Watts to about 100 Watts for a period of from about 5 seconds to about 30 minutes.

11. The method of claim 1 wherein the solid substrate is a material selected from the group consisting of: a plastic, gold or silver metal.

12. The method of claim 1 wherein the siloxane film is formed from tetraorthosilicate (TEOS) or hexamethyldisiloxane (HMDSO) or a mixture thereof.

13. The method of claim 1 wherein the chemical functionality precursor is a compound having a chemical functionality selected from the group consisting of: carboxyl, ether, acrylic acid, acetic acid, acetaldehyde, formaldehyde, amines, ethers, thiols and per-fluoro functionality.

14. The method of claim 13 wherein the compound is a carboxylic acid, an acrylic acid (AA), an acetic acid, an acetaldehyde or formaldehyde.

15. The method of claim 13 wherein the ether arises from co-deposition using a volatile glycol-ether precursor, such diethylene glycol dimethylether (DEGDME).

16. The method of claim 14 wherein the compound having a chemical functionality is acrylic acid and the deposition of step (ii) is for about 15 seconds, and the deposition step of (iii) is for about 30 seconds.

17. The method of claim 16 wherein the siloxane precursor is selected from the group consisting of: aminopropyltriethoxysilane (APTES), mercaptopropyltriethoxysilane (MPTES), 3-glycidoxypropyltrimethoxysilane (GOPTMS), perfluoroctyltriethoxysilane (FOTES).

18. The method of claim 1 wherein the siloxane precursor and the chemical functionality precursor are the same and may be selected from the group consisting of: aminopropyltriethoxysilane (APTES), mercaptopropyltriethoxysilane (MPTES), 3-glycidoxypropyltrimethoxysilane (GOPTMS), perfluoroctyltriethoxysilane (FOTES).

19. The method of claim 1 further comprising the step of adding an oxygen or carbon source to the plasma during at one of the steps (i) to (iii).

20. The method of claim 1 further comprising a masking step wherein a portion of the substrate surface is masked to facilitate substrate surface patterning.

21. The method of claim 1 where the operating pressure range from about 80 mTorr to about 600 mTorr

22. The water swellable solid substrate coating obtained directly from the method of claim 1.

23. A coating for a solid substrate, the coating comprising:
a first siloxane layer; and
a second layer comprising a chemical functionality
wherein the first and second layers are plasma polymerised onto the substrate, the coating swellable on contact with an aqueous solution by factor of at least 2, based on RMS (root mean square) roughness swelling measurement.

24. The coating of claim 23 wherein the coating provides a ratio of specific to non specific analyte binding in bioassays of greater that 2 as detected using fluorescent detection.

25. The coating of claim 23 wherein the chemical functionality film provided on top of the first surface film is selected from the group consisting of: carboxyl, ether, acrylic acid, acetic acid, acetaldehyde, formaldehyde, amines, ethers, thiols and per-fluoro functionality.

26. The coating of claim 25 wherein the chemical functionality comprises carboxyl.

27. The coating of claim 26 wherein the carboxyl functionality contains the carbonyl group, wherein the carbonyl groups are in the range of from about 0.5 to about 50% and where the content of carbon in said coating is in the range of 1-86 atomic % as determined by XPS.

28. The coating of claims of claim 25 wherein the carboxyl is derived from an acrylic acid precursor.

29. The coating of claim 23 wherein the siloxane forms a graded siloxane network which interlinks with the chemical functionality layer.

30. The coating of claim 23 wherein the solid substrate is selected from the group consisting of a plastic, a gold or silver metal.

31. The coating of claim 23 wherein the first and second layers have a total combined layer thickness in the range of about 10 angstroms to about 1000 angstroms.

32. The coating of claim 23 wherein the coating exhibits a water contact angle of from about 5 degree to about 60 degrees.

33. The coating of claim 23 wherein the RMS roughness is precisely measured by atomic force microscopy (AFM).

33. A product comprising the solid substrate having coating of any one of claims 22 to 31.

34. Use of a coating for a solid substrate the coating comprising:
a first siloxane layer; and
a second layer comprising a chemical functionality wherein the first and second layers are plasma polymerized onto the substrate, the coating swellable on contact with an aqueous solution by factor of at least 2, based on RMS (root mean square) roughness swelling measurement to immobilize an analyte in a biodiagnostic application, such as an immunoassay.

35. Use of a water swellable coating for a solid substrate the coating comprising:
a first siloxane layer; and
a second layer comprising a chemical functionality
wherein the first and second layers are plasma polymerized onto the substrate, the coating swellable on contact with an aqueous solution by factor of at least 2, based on RMS (root mean square) roughness swelling measurement as a tool for biological discovery and/or biomedical detection, in medical imaging and/or therapeutic applications such as cell labeling, targeted drug delivery, targeted gene delivery, biosensing, cell separation, cell purification and imaging.
